(19) United States
(12) Patent Application Publication
(10) Pub. No.: US 2026/0114765 A1
(43) Pub. Date: Apr. 30, 2026

(54) MINIATURIZED CONTINUOUS TRANSCUTANEOUS CARBON DIOXIDE AND PERIPHERAL OXYGEN SATURATION MONITORING WEARABLE

(71) Applicant: Worcester Polytechnic Institute, Worcester, MA (US)

(72) Inventors: Ulkuhan Guler, Shrewsbury, MA (US); Tuna B. Tufan, Worcester, MA (US); Isil Isiksalan, Worcester, MA (US)

(21) Appl. No.: 19/368,711

(22) Filed: Oct. 24, 2025

Related U.S. Application Data
(60) Provisional application No. 63/711,995, filed on Oct. 25, 2024.

(51) Int. Cl. A61B 5/1455; A61B 5/00

(52) U.S. Cl. CPC A61B 5/14552; A61B 5/6832; A61B 5/7271; A61B 2560/0462; A61B 2562/0233

(57) ABSTRACT

A wearable, skin adhesive blood gas monitoring device detects both oxygen and carbon dioxide for accommodating a potential inaccuracy in an oxygen level resulting from excessive carbon dioxide. Photoluminescent films in conjunction with a compact form-factor circuit provide a wearable and unobtrusive device that adheres directly to the epidermal surface for computation of blood gas levels based on transcutaneous and reflective properties of the blood gases. A wireless medium such as Bluetooth® enables communication to a standalone or portable device such as a smartphone or similar personal device having an application (app) for receiving the computed signals. Conditions such as hypercarbia, which tend to result in higher levels of carbaminohemoglobin, or hemoglobin bound to CO₂ instead of oxygen, tend to compromise computed oxygen levels by interpreting the CO₂-bound hemoglobin as oxygenated blood.

### RELATED APPLICATIONS

This patent application claims the benefit under 35 U.S.C. § 119(e) of U.S. Provisional Patent App. No. 63/711,995, filed Oct. 25, 2024, entitled “MINIATURIZED CONTINUOUS TRANSCUTANEOUS CARBON DIOXIDE AND PERIPHERAL OXYGEN SATURATION MONITORING WEARABLE,” incorporated herein by reference in entirety.

### STATEMENT REGARDING FEDERALLY SPONSORED RESEARCH AND DEVELOPMENT

This invention was developed with U.S. Government Support under contract No. ECCS-2143898, awarded by the National Science Foundation (NSF), and contract No. 1R01HL172293-01, awarded by the National Institute for Health (NIH). The Government has certain rights in the Invention.

### BACKGROUND

Blood gas testing is an effective tool for analysis and diagnosis of many human physiological and conditions. Among the vital signs of human body, respiration is a key component of a person's health. Respiratory health can be quantified by rate, volume, and blood-gas content. Traditional respiration monitoring methods such as arterial blood gas monitoring and pulse oximetry have certain advantages and disadvantages. Blood gas monitors tend to be either bulky, stationary devices and/or require a tether to a patient, limiting effectiveness in tracking an ambulatory patient in a non-clinical setting.

### SUMMARY

A wearable, skin adhesive blood gas monitoring device detects both oxygen and carbon dioxide for accommodating a potential inaccuracy in an oxygen level resulting from excessive carbon dioxide. Photoluminescent films in conjunction with a compact form-factor circuit provide a wearable and unobtrusive device that adheres directly to the epidermal surface for computation of blood gas levels based on transcutaneous and reflective properties of the blood gases. A wireless medium such as Bluetooth® enables communication to a standalone or portable device such as a smartphone or similar personal device having an application (app) for receiving the computed signals. Conditions such as hypercarbia, which tend to result in higher levels of carbaminohemoglobin, or hemoglobin bound to CO₂ instead of oxygen, tend to compromise computed oxygen levels by interpreting the CO₂-bound hemoglobin as oxygenated blood.

Configurations herein are based, in part, on the observation that blood gas monitoring is an effective parameter in treatment of respiratory and also other health conditions, and is typically monitored as a standard practice in healthcare settings, often continuously. Unfortunately, conventional blood gas monitoring techniques, such as pulse oximetry, suffer from the shortcoming that they tend to ignore the often small contribution of CO₂ bound hemoglobin to the computed oxygen level. While sometimes the CO₂-bound hemoglobin is negligible or represents an acceptable error, in some patients, particularly those with hypercarbia, the computed oxygen level can be compromised significantly.

Accordingly, configurations herein substantially overcome the shortcomings of conventional, tethered blood gas monitoring by simultaneously computing a carbon dioxide level along with the oxygen level for allowing true blood gas levels to be determined by considering the CO₂ level compromising or skewing the computed oxygen reading. A miniaturized circuit and epidermally adhered luminescent film provide a wearable, untethered device configured for computing transcutaneous carbon dioxide (PtcCO₂) and peripheral blood oxygen saturation (SpO₂) from a single processor with adjacent photoluminescent sensors encapsulated in a skin mounted form. Battery power and a Bluetooth or similar transmission medium allow communication with a tracking app or device for storing and alerting appropriate parties in the event of problematic readings. In this manner, the disclosed approach overcomes limitations of traditional carbon dioxide monitors which typically require a heating element and large, bedside monitor that prevents continuous monitoring outside a clinical setting.

In further detail, configurations disclosed herein present an epidermally mounted wearable blood gas monitoring device, system and method employing an epidermally mounted substrate, which includes a luminescent measurement circuit configured for computing a carbon dioxide blood gas level, and a reflective circuit configured for computing an oxygen blood gas level. A processor or microcontroller effects simultaneous computation of transcutaneous carbon dioxide (PtcCO₂) and peripheral blood oxygen saturation (SpO₂), and an epidermal engagement medium such as a patch or adhesive provides gaseous communication with a sensed epidermal surface for detection of oxygen and carbon dioxide.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a patient in an ambulatory monitoring environment using the disclosed wearable blood gas monitoring device;

FIG. 2 shows a graphical illustration of CO2 bound hemoglobin emulating hemoglobin bound oxygen;

FIG. 3 shows a schematic of a side view of the wearable blood gas monitoring device engaged with a patient epidermal medium;

FIGS. 4A and 4B show fluorescence and phosphorescence of light passing through the epidermal engagement medium of FIGS. 1-3;

FIGS. 5A-5B show luminescence levels for computation in the luminescent measurement circuit for CO₂ sensing; and

FIG. 6 shows a data collection processing flow of the wearable blood gas monitoring device of FIGS. 1-5B.

### DETAILED DESCRIPTION

The description below presents an example of a miniaturized wearable device, such as a patch or a smartwatch, that simultaneously monitors transcutaneous carbon dioxide (PtcCO₂) and peripheral blood oxygen saturation (SpO₂). An advantage of integrating both sensing modalities on a single printed circuit board (PCB) and performing real-time, on-board data processing, is eliminating the need for multiple devices or external processors.

For PtcCO₂ monitoring, the device uses a dual-luminophore luminescent sensing film. One luminophore is CO₂-sensitive, while the other serves as a reference to cancel out environmental noise. The light emitted by the film is detected using a photodiode, and the time-domain dual lifetime referencing (t-DLR) technique ensures accurate measurements by compensating for external factors.

For SpO₂, the device employs pulse oximetry with red LED that measure the absorption of light by oxygenated and deoxygenated hemoglobin, allowing it to calculate oxygen saturation levels. The same LED is also used to gather heart rate and respiratory rate data. The device's microcontroller unit (MCU) processes both PtcCO₂ and SpO₂ data in real time, eliminating the need for off-board post-processing, reducing complexity, and optimizing power consumption. The wearable will be flexible and lightweight, allowing for comfortable, long-term use, particularly for neonates, children, and adults with respiratory conditions like COPD. Power is provided by a small rechargeable battery or a USB (Uniform Serial Bus) connector for extended operation.

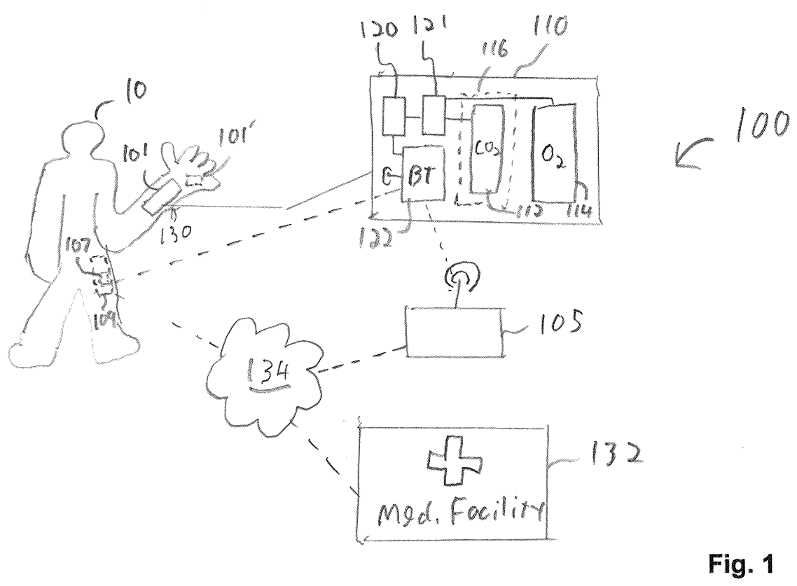

FIG. 1 is a context diagram of a patient 10 in an ambulatory monitoring environment 100 using the disclosed wearable blood gas monitoring device 101. The epidermally mounted wearable blood gas monitoring device 101 integrates an epidermally mounted substrate 110 with a luminescent measurement sensor 112 configured for computing a carbon dioxide blood gas level. Also on the substrate 110 is a reflective sensor 114 configured for computing an oxygen blood gas level. An analog front end (AFE) 121, processor 120, microcontroller and/or memory allows for simultaneous computation of transcutaneous carbon dioxide (PtcCO₂) and peripheral blood oxygen saturation (SpO₂). The AFE receives analog signals from the sensors 112, 114, typically a voltage or current level, and sends digital singals to the processor 120, An epidermal engagement medium 130 such as an adhesive or strap maintains the substrate 110 in gaseous communication with the sensed epidermal surface for detection of oxygen and carbon dioxide.

A particular advantage is the luminescent measurement sensor 112 and the reflective sensor 114 disposed in an adjacency on the epidermal engagement medium 130 and coupled to the AFE 121 and processor 120. A luminescent film 116 is in contact with the epidermal surface for receiving transcutaneously passed gases (CO₂), while the substrate 110 is layered on top of the film for effecting LED projection of light, discussed further below.

The device 101, when enabled with wireless communication through Bluetooth Low Energy (BLE) or similar protocols enables continuous transmission of real-time data from a transmitter 122 to a base station 105 or smart device 109, can define a system where healthcare providers can remotely monitor patients' respiratory health. An app 107 on the smartphone or the base station can achieve connectivity to a public access network 134 such as the Internet for communication and downloads to a healthcare facility 132 or provider. The device 101 will also store data locally for further analysis, helping medical professionals track long-term trends and make informed decisions about treatment. This wearable solution offers a complete, noninvasive monitoring system that integrates both PtcCO₂ and SpO₂ measurements, significantly advancing respiratory health monitoring in hospital and home care environments. Particular advantages are apparent to COPD (chronic obstructive pulmonary disease) patient and neonatal respiratory deficiencies.

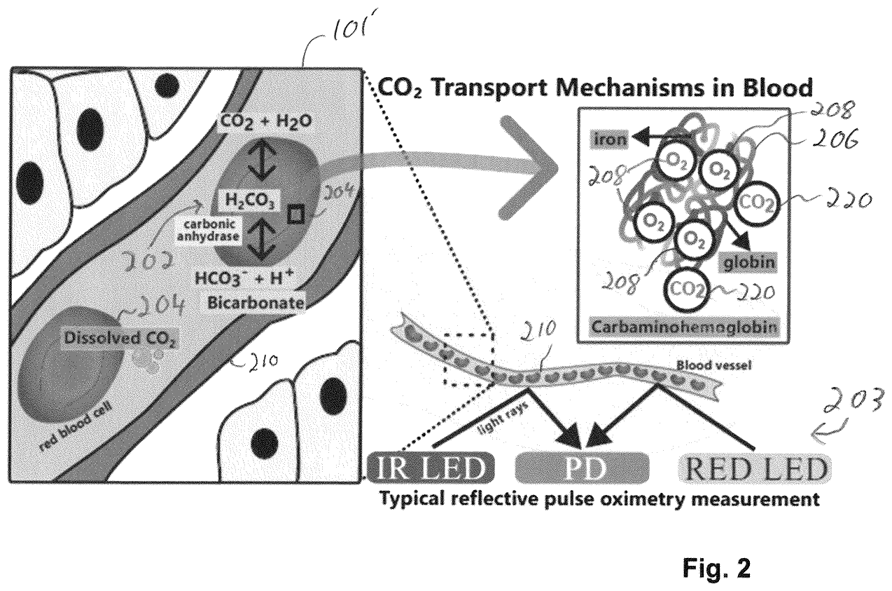

FIG. 2 shows a graphical illustration of CO₂ bound hemoglobin emulating hemoglobin bound oxygen. Referring to FIG. 2, in a particular use case. A major challenge in COPD management is the occurrence of exacerbations, defined as sudden worsening of symptoms that may require hospitalization. Remote patient monitoring (RPM) has emerged as a promising strategy for enhancing COPD management. However, while some existing RPM systems have shown promising results, they primarily rely on monitoring peripheral oxygen saturation (SpO₂), heart rate (HR), and respiration rate (RR), which are all derived from the underlying photoplethysmogram (PPG) signal. These sensors provide only basic vital parameter screening and do not directly measure changes in ventilation or the corresponding increase in CO₂, which is a hallmark of COPD exacerbations. Additionally, SpO₂ measurements in COPD patients may be inaccurate in the presence of hypercarbia (elevated CO₂) due to additional CO₂ binding, illustrated in FIG. 2, leading to misleading SpO₂ readings.

FIG. 2 shows a patient rendering 101′ of a simplified CO₂ transport mechanism 202 with a hemoglobin 206 binding visualization, along with a typical reflective SpO₂ measurement 203. Basic physiology establishes that blood vessels 210 transport red blood cells 204 having hemoglobin 206 for binding with oxygen 208. Elevated levels of CO₂ 220 can cause the normally inconsequential CO₂ bound hemoglobin (carbaminohemoglobin) to “masquerade” as oxygenated hemoglobin, leading to inaccurate oxygen readings.

The disclosed approach presents a device capable of concurrently measuring SpO₂ and CO₂ levels along with heart rate and respiration rate. This device has the potential to identify the inaccuracies of SpO₂ measurements caused by elevated CO₂. The device 101 determines a carbon dioxide level erroneously contributing to the sensed SpO₂ by computing the blood oxygen saturation based on both oxygen and carbon dioxide bound to hemoglobin, and then determining, based on the carbon dioxide blood gas level, a portion of the computed oxygen blood gas level inaccurately attributed to carbon dioxide bound to hemoglobin. The analysis circuit and processor then compute the peripheral blood oxygen saturation based on an adjustment for the inaccurately attributed carbon dioxide bound to hemoglobin.

Respiratory issues and disease such as COPD tend to cause CO₂ retention. Conventional CO₂ monitoring can be invasive and time consuming. To overcome such limitations, transcutaneous CO₂ (PtcCO₂) monitoring provides a noninvasive alternative, which estimates PaCO₂ by measuring CO₂ diffusion through the skin and offers a viable solution for noninvasive patient assessment.

Although CO₂ information offers valuable insights, it alone does not provide a complete picture of respiratory health. Oxygenation status is equally critical. Pulse oximetry has been established as a noninvasive method for estimating hemoglobin saturation of arterial oxygen (SaO₂). By measuring the absorption of red and infrared light by oxygenated and deoxygenated hemoglobin, pulse oximeters provide an estimate known as SpO₂. While widely used for continuous oxygen monitoring, SpO₂ has notable limitations, including in individuals with COPD.

Determinations of SpO₂ can overestimate SaO₂ in the presence of hypercarbia, leading to artificially inflated reading levels. In hypercarbic conditions, levels of carbaminohemoglobin (HbCO₂), the normally small amount of hemoglobin carrying CO₂ increases from approximately 5% to up to 30%, leading to falsely elevated SpO₂ readings by distorting the light absorption of hemoglobin, as illustrated in FIG. 2. Additionally, CO₂ levels impact the interpretation of SpO₂ since the ensuing acidosis shifts the oxygen dissociation curve, altering the relationship between PaO₂ and SpO₂.

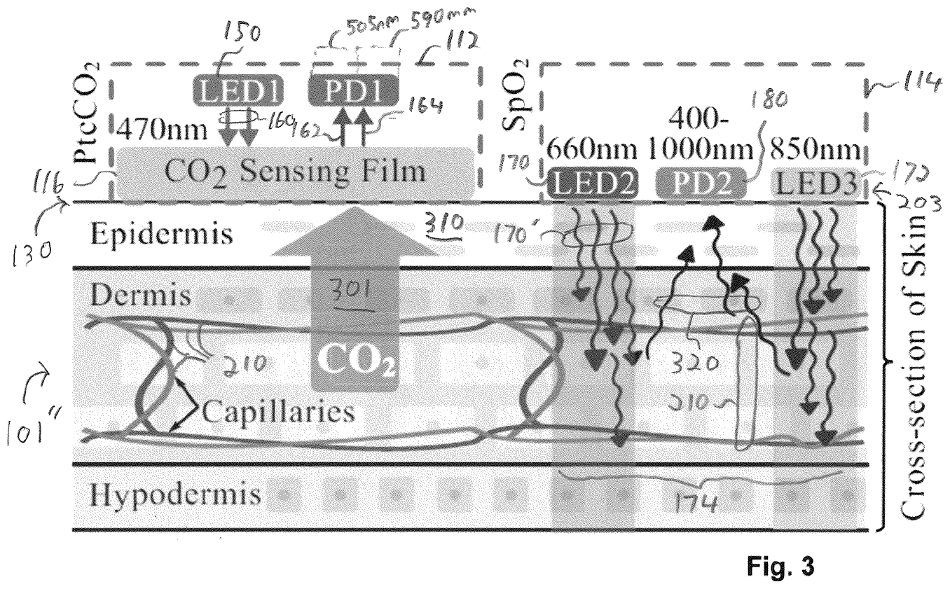

FIG. 3 shows a schematic of a side view of the wearable blood gas monitoring device engaged with a patient epidermal medium. By integrating PtcCO₂ monitoring with SpO₂, as depicted in FIG. 3, the disclosed device provides a real-time, dual-parameter assessment of both CO₂ levels and SpO₂ readings. If PtcCO₂ surpasses a critical threshold, the device 101 can flag potential inaccuracies in SpO₂, prompting timely clinical reassessment. This integrated approach bridges the gap between noninvasive home monitoring and clinical-grade ABG (Arterial Blood Gas) assessment, offering a more reliable respiratory monitoring solution for COPD patients. Additionally, by providing real-time CO₂ trends, the disclosed approach supports future research on SpO₂ correction algorithms, enhancing oxygen saturation assessments and improving clinical decision-making in long-term respiratory care.

Referring to FIG. 3, the luminescent measurement sensor 112 and the reflective sensor 114 are shown with the epidermal engagement medium 130 disposing the luminescent film 116 and reflective SpO₂ measurement 203 on the skin surface. The patient cross section 101″ shows that the epidermal engagement medium 130 transports and/or receives CO₂ 301 from a gaseous emission of an epidermal surface 310 and receives an indication of O₂ based on a blood vessel 210 flow 320 beneath the epidermal surface 310. The adjacently located luminescent measurement sensor 112 and the reflective sensor 114 for PtcCO₂ and SpO₂, respectively, allows for gathering and computations involving both of these related parameters.

The luminescent measurement sensor 112 further includes the luminescent film 116 responsive to a predetermined wavelength of light, and an illumination source 150 configured to emit light 160 in the predetermined wavelength. The luminescent film 116 is responsive to the illumination source for emitting light of a responsive wavelength based on a concentration of carbon dioxide.

In operation, the luminescent film 116 emits a CO₂ sensitive emission signal 162 and a reference emission signal 164 in response to the transcutaneous carbon dioxide 301. The processor 120 compares the CO₂ sensitive emission signal 162 to the reference emission signal 164 for determining the level of transcutaneous carbon dioxide. A particular example luminescent measurement sensor 112 is configured for performing a dual-lifetime referencing (DLR) technique based on the predetermined wavelength in the range of 465-475 nm, and the responsive wavelength includes a 500-510 nm wavelength and a 585-595 nm wavelength, discussed further below.

Turning to the oxygen sensing for computing an initial SpO₂ level, the reflective sensor 114 further includes an illumination source 170 having a wavelength based on a first hemoglobin sensitivity, and an illumination source 172 having a wavelength based on a second hemoglobin sensitivity. The illumination sources 170, 172 for the first and second hemoglobin sensitivities are directed towards a common epidermal location 174 for reflecting energy (light/photons) at an intensity based on absorption by the blood cells 204 in the vessels 210. A photodetector 180 is disposed opposed from the common epidermal location 174 and is positioned to receive the reflected light 320 indicative of an oxygen saturation ratio.

In the example configuration, the oxygen saturation ratio is based on a ratio of reflected light corresponding to the first and second hemoglobin sensitivities, where the first and second hemoglobin sensitivities are differentiated based on an oxygenation state of the hemoglobin molecules 206. The projected light 170′, 172′ interacts with oxygenated hemoglobin (HbO2) and deoxygenated hemoglobin (RHb) in the blood. The reflected light 320 therefore has an intensity in wavelengths corresponding to oxygenation states of hemoglobin in the blood vessels 210 beneath the common epidermal location 174, and the oxygen saturation ratio is based on absorption of hemoglobin corresponding to the respective oxygenation states, discussed further below. A particular example reflective sensor 114 is configured for emitting the illumination source in a wavelength of 655-665 nm (170) and a wavelength of 845-855 nm (172), and the photodetector 180 is responsive to the reflected light in a wavelength between 400 nm and 1000 nm.

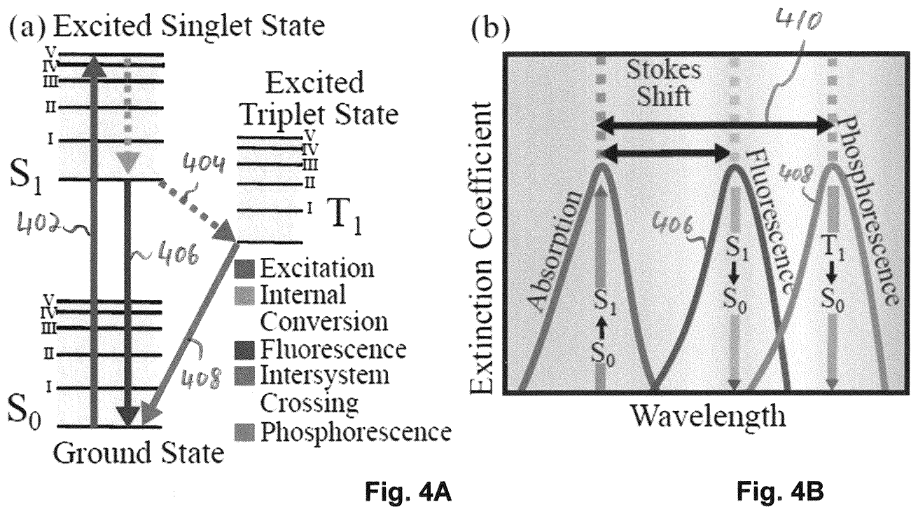

FIGS. 4A and 4B show fluorescence and phosphorescence of light passing through the epidermal engagement medium of FIGS. 1-3. Referring to FIGS. 4A and 4B, The PtcCO₂ sensing circuit 112 preferably utilizes a luminescent film SP-CD1-D5-rMy-US sourced from PreSens™ Precision Sensing, of Regensburg, Germany. Any suitable luminescent film may be provided in alternate configurations. Configurations herein leverage the luminescent properties of this film for the transcutaneous CO₂ sensing, as illustrated in the Jablonski diagram shown in FIG. 4A, which depicts the photo-physical processes involved in absorption and emission. The singlet ground and first electronic states are depicted as S₀ and S₁, respectively. At each of these electronic energy levels, luminophores can exist in multiple vibrational energy levels, depicted as I, II, III, etc. Following an excitation, a luminophore is usually excited 402 to a higher level of S₁. With few exceptions, molecules in condensed phases rapidly relax to the lowest level of S₁, a process known as internal conversion. These internal transitions are nonradiative and vibrational. Molecules in S₁ return to the ground state, S₀, through a process called fluorescence 406. This process typically happens on the nanosecond timescale and yields photons of longer wavelength (lower energy) than the excitation light due to energy losses during vibrational relaxation and internal conversion. Molecules in the S₁ state may also undergo spin conversion 404 to the first triplet state, T₁. Emission from T₁ is termed phosphorescence 408 and generally appears at longer wavelengths compared to fluorescence. The conversion from S₁ to T₁ is known as intersystem crossing. The energy of emission in both types of transitions is typically lower than that of absorption. This energy loss between excitation and emission, known as the Stokes shift 410, is a universal characteristic of luminescent molecules. A simplified visualization of the Stokes shift can be seen in FIG. 4B. To clarify the terminology, luminescence refers to the spontaneous emission of light by a substance following the absorption of energy. Luminescence encompasses radiative processes such as fluorescence 406, which originates from singlet states, and phosphorescence 408, which originates from triplet states, as described above.

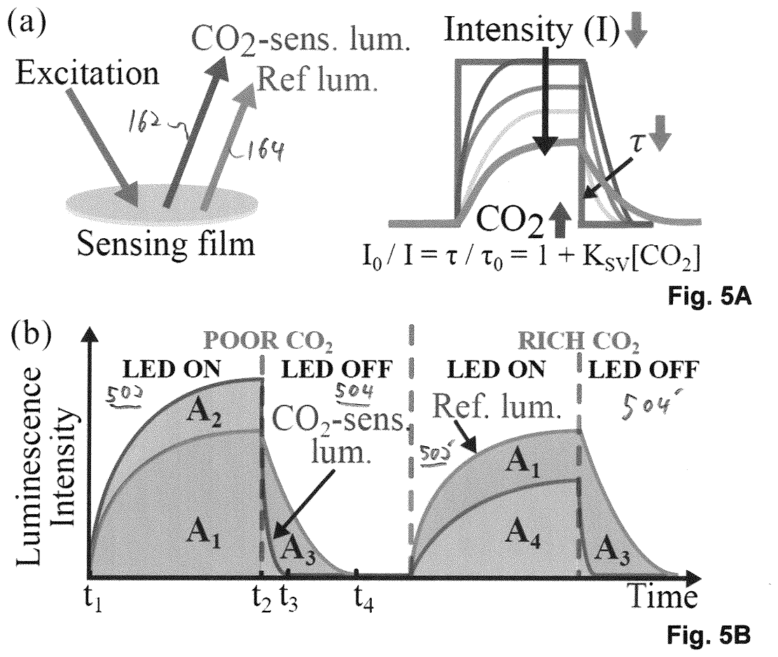

FIGS. 5A-5B show luminescence levels for computation of the luminescent measurement sensor for CO₂ sensing. The luminescent film 116 has dual luminophores, such that the dual luminophores are selectively responsive to emit a CO₂ sensitive emission signal and a reference emission signal, wherein a difference in the CO₂ sensitive emission signal and the reference emission signal varies between an on and off state of the illumination source. The luminescent film 116, or sensing film contains two types of luminophores: a CO₂-sensitive fluorescent luminophore (fluorophore) and a reference phosphorescent luminophore (phosphor). The fluorophore exhibits changes in both fluorescence intensity and lifetime in response to CO₂ fluctuations, while the phosphor remains unaffected by such changes. Upon excitation with a 470 nm blue light source, the film generates two emission signals: a CO₂-sensitive fluorescence 162 at 505 nm and a stable reference phosphorescence 164 at 590 nm, as shown in FIG. 5A. Other wavelengths may be sensed, depending on a relationship with the luminescent film and gaseous-based excitation. Intensity-based luminescence measurements are highly susceptible to variations in the optical path, such as scattering, motion, or alignment shifts, which are common in wearable health sensors. In contrast, lifetime- or ratiometric-based methods provide more robust and reliable readings by minimizing these artifacts. Having phosphorescent reference molecules integrated into the luminescent film 116 enables the quantification of fluorescence response using a ratio-metric technique, the dual-lifetime referencing (DLR) technique, which can be applied in both the time and frequency domains. In the frequency-domain method, the fluorescence response is transformed into a phase shift, providing a readout signal that is resistant to interference, and thus enabling more accurate readings. In the time-domain approach, the luminescence material is periodically excited using square light pulses. The resulting emission is recorded in two distinct time windows: one during the LED ON phase 502 and another during the LED OFF phase 504. The ratio of the signals from these windows enables accurate calculation of fluorescence response. This approach, illustrated in FIG. 5B, defines time-domain DLR (t-DLR).

The LED ON window 502 represents the combined intensity of the CO₂-sensitive fluorophore and the reference phosphor during excitation. When CO₂ levels are low, the CO₂-sensitive fluorophore (A₂) exhibits higher intensity than the reference phosphor (A₁), resulting in a total LED ON area of A₁+A₂. Conversely, at higher CO₂ levels, the CO₂-sensitive fluorophore decreases (A₄), reducing the total LED ON area 502′ to A₁+A₄. If we denote the area during the LED ON window as Aon and the area during the LED OFF 504′ window as A_{OFF}, these regions can be formulated as follows:

$${A}_{ON} = {C}_{EXT} ( \frac{{L}_{{SEN}_{0}}}{{P CO}_{2}} {\int }_{t} {L}_{SEN - ON} ( t ) dt + {L}_{{REF}_{0}} {\int }_{t} {L}_{REF - ON} ( t ) dt )$$

where C_{EXT} represents interfering factors, L_{SEN}0 and L_{REF}0 are luminescence constants, PCO₂ is the partial pressure of CO₂ in the environment, and L_{SEN-ON}(t) and L_{REF-ON}(t) denote the intensity of the respective CO₂-sensitive fluorophore and reference phosphor in the time domain.

The LED OFF region, seen in FIG. 5B, captures the decay of luminescence after the LED is turned OFF, primarily reflecting the activity of the reference phosphor. In contrast, the sensitive fluorophore decays rapidly within the nanosecond range. This presents a challenge for low-cost, power-efficient devices, as capturing the nanosecond-scale decay requires high-speed optical acquisition systems as well as high-speed electronic readouts for proper sampling. Unlike the excitation phase, the A_{OFF} region primarily reflects the reference phosphor's decay (A₃), which remains stable across varying CO₂ levels. Since the reference phosphor decays within the microsecond range, its contribution to the integrated A_{OFF} region is significantly larger. A_{OFF} region is calculated:

$${A}_{OFF} = {C}_{EXT} \cdot {L}_{{REF}_{0}} ( {\int }_{t} {L}_{REF - OFF} ( t ) )$$

where L_{REF}0 is a luminescence constant, and LREF-OFF(t) represents the intensity of the reference phosphor during the decay phase in the time domain. By taking the ratio of the two integrated regions, referred to as the luminescence ratio (LR), we obtain a measurement of CO₂ that is robust against confounding factors such as excitation source intensity and detector photosensitivity, as the term C_{EXT} cancels out, as shown below:

$$LR = \frac{{A}_{ON}}{{A}_{OFF}} = \frac{\frac{{L}_{{SEN}_{0}}}{{P CO}_{2}} \cdot ( {\int }_{t} {L}_{SEN - ON} ( t ) ) + {L}_{{REF}_{0}} \cdot ( {\int }_{t} {L}_{REF - ON} ( t ) )}{{L}_{{REF}_{0}} ( {\int }_{t} {L}_{REF - OFF} ( t ) )}$$

The oxygen sensing circuit complements the CO₂ sensing circuit by providing the initial, or base, oxygen level prior to computing undue influence from the CO₂ level. Pulse oximetry is a widely used noninvasive technique for measuring SpO₂. It operates by emitting red and infrared (IR) light from light-emitting diodes (LEDs) onto the skin, where the light interacts with oxygenated hemoglobin (HbO₂) and deoxygenated hemoglobin (RHb) in the blood. Due to their distinct optical properties, these hemoglobin states absorb red and IR light differently, allowing for differentiation between oxygenated and deoxygenated blood. The unabsorbed light is reflected and detected by a photodiode (PD), as illustrated in FIG. 3.

By analyzing the intensity of the reflected light at specific wavelengths, i.e., 600 nm and 850 nm, the system calculates the ratio of red and IR absorption, which is then used to determine SpO₂. This calculation is based on the ratio R:

$$R = [ \frac{{AC}_{RED}}{{DC}_{RED}} ] / [ \frac{{AC}_{IR}}{{DC}_{IR}} ] ,$$

where R represents the SpO₂ ratio, a.k.a. oxygen saturation ratio, and AC_{RED}, AC_{IR}, DC_{RED}, and DC_{IR} correspond to the alternating current (AC) and direct current (DC) com-ponents of the red and IR signals, respectively. The AC component reflects pulsatile blood flow, while the DC component represents non-pulsatile blood volume. Once R is determined, it is mapped to an SpO₂ value through an empirical calibration curve. This mapping depends on the LED wavelengths, detector characteristics, and optical path. A commonly used form is linear, given by:

$${Sp O}_{2} = A - B \cdot R ,$$

where A and B are calibration constants. In particular configurations, it was found that values of A=117 and B=25 provided reliable estimations for SpO₂ in agreement with reference measurements.

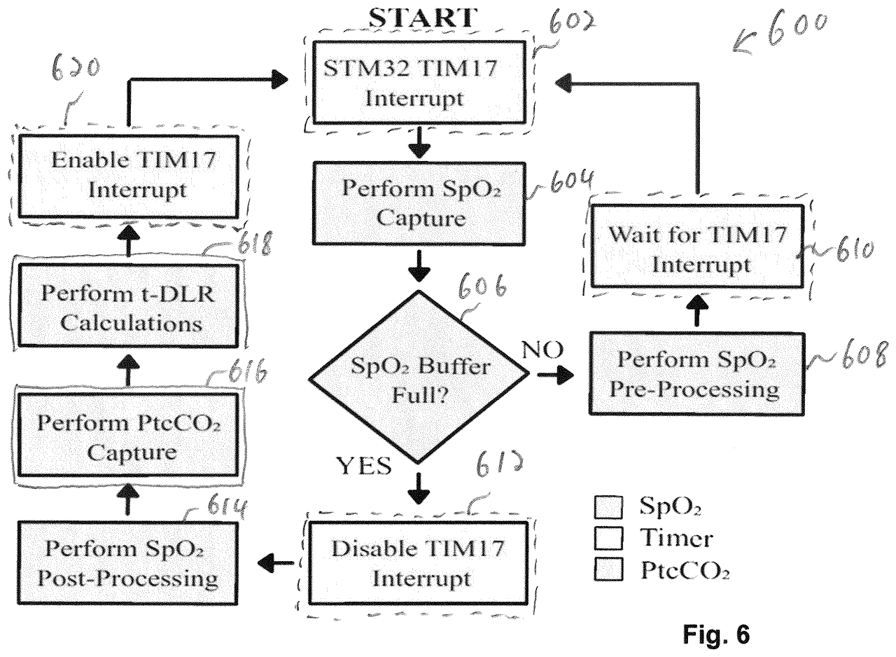

FIG. 6 shows a data collection processing flow of the wearable blood gas monitoring device of FIGS. 1-5B. In a particular example implementation, the dual-mode sensing enables concurrent measurement of two modalities, i.e., PtcCO₂ and SpO₂, where PtcCO₂ sensing is enabled by de-ploying a green-sensitive photodiode PD1 (SD019-141-411-G, available commercially from Advanced Photonix) to detect luminescence emitted from the sensing film accurately. The excitation source, LED1 (LXZ1-PR0 by Lumileds) provides blue light excitation to the sensing film. The SpO₂ sensor head part integrates a dual-wavelength LED module (VSMD66694 by Vishay) for red and infrared emissions, designated as LED2, and LED3, alongside the photodiode PD2 (S13773 by Hamamatsu), as illustrated in FIG. 3. Other vendors may of course be sought for implementation of alternative configurations.

The signal acquisition hardware performs acquisition and processing managed by the ADPD4100 (commercially available from Analog Devices Inc.), a highly integrated photometric front-end IC designed for luminescence- and reflectance-based sensing. The chip includes 8 LED drivers, 8 input channels with programmable transimpedance amplifiers (TIAs), filters, and a 14-bit analog to digital converter (ADC), an onboard first-in first-out (FIFO) buffer, accessible via inter-integrated circuit (I²C) or serial peripheral interface (SPI). In the PtcCO₂ configuration, the ADPD4100 operates in impulse response mode, optimized for luminescence capture. For SpO₂, the configuration is similar, but the RED and IR LEDs are pulsed at different times using the device's timeslot feature, which enables multiple captures within a single sequence for distinguishing signals from different sources. During operation, the PtcCO₂ sensing film is first excited by LED1, after which the emitted luminescence is detected by PD1 and an optical filter to suppress excitation leakage. The resulting photocurrent is converted to voltage via the TIA, filtered, and then digitized by the ADC before being stored in the FIFO. Processor 120, implemented by a microcontroller (MCU) then retrieves this data over I²C, while also controlling system timing, configuration, and acquisition flow. A power management unit (PMU) generates and distributes regulated supplies—5 V for the LED, 3.3 V for the MCU and an analog front-end (AFE) I/O, and 1.8 V for the AFE's analog/digital blocks—derived from a single 3 V coin cell using charge pumps and a low dropout regulator (LDO).

Referring to FIGS. 1-6, FIG. 6 depicts the logic flow and onboard calculations 600 for both the t-DLR technique in PtcCO₂ measurements and pulse oximeter-based SpO₂ measurements are executed on the same MCU. This process is managed by an internal timer, configured to trigger at 36 Hz to coordinate SpO₂ acquisition, designated by dashed outlines in FIG. 6. Upon each timer interrupt at step 602, the MCU first initiates SpO₂ data capture from the AFE and reads the 32-bit words from its FIFO (4-byte FIFO configuration in standard sampling mode), as depicted at step 604. For SpO₂, two timeslots operate at 250 Hz so IR and red are captured in an interleaved sequence; each slot uses 8 LED pulses per capture with a 2 us pulse width. The FIFO delivers the timeslot order-first 32-bit word IR, second 32-bit word RED—and the MCU stores these into a circular buffer such that even indices hold IR and odd indices hold RED. The buffer includes full and empty flags, holds 2,050 elements, and fills in about 8 s at 250 Hz. A check is performted, at step 606, and if the buffer is not full, the MCU performs light preprocessing such as filtering and downsampling, as disclosed at step 608, then waits for the next interrupt, as shown at step 610. When the buffer becomes full, the timer interrupt is temporarily disabled, as depicted at step 612, and SpO₂ post-processing is executed at step 614. The system then performs a PtcCO₂ acquisition (solid double outlined boxes), at step 616, reading 2-byte samples from the impulse-response FIFO (14-bit ADC path), applies t-DLR, as depicted at step 618, and re-enables the timer to resume the SpO₂ loop, as shown at step 620.

Within this processing loop 600, the PtcCO₂ path requires finer sampling of the decay waveform for accurate lifetime estimation. The impulse response mode of the ADPD4100 provides a maximum sampling rate of 2 μs, which introduces additional error when calculating the luminescence decay time. To overcome this hardware limitation, two nearly identical optical acquisition channels were configured, with the second delayed by 1 μs. Combining the data from both channels, achieves an effective sampling rate of 1 μs. This simple approach allowed us to overcome the AFE's sampling rate constraint and achieve adequate resolution for accurate decay curve fitting. In the disclosed configuration, the CO₂ excitation uses a 32 μs LED pulse at 100 mA, which is found to be sufficient while limiting photobleaching; the resulting decay occurs on the order of several microseconds. At 1 μs effective sampling, the analyzed decay window contains ˜8 samples.

In actual patient operation, continuous physiological monitoring benefits from real-time on-board computations to minimize latency while maintaining low power consumption. To achieve this, a regression-based approach was implemented for estimating luminescence lifetime, beneficial for the t-DLR technique. The computation logic reconstructs an exponential decay through a set of linear operations, providing a computationally lightweight alternative to iterative optimization methods.

Luminescence lifetime, denoted as t, is the characteristic time it takes for a molecule to return from an excited state to a lower-energy state via photon emission. In luminescence-based sensing, particularly in the t-DLR technique, an accurate determination of τ is significant, as it directly influences the reliability of CO₂ measurements. The lifetime is extracted by fitting the recorded luminescence decay to an exponential model:

$$Output = {\underbrace{α}}_{scaling factor} {e}^{- τ t} + {\underbrace{Δ}}_{D C offset} ,$$

where t represents the given time, α is a scaling factor, and Δ is the DC offset. A closed-form analytical regression method is employed to estimate r efficiently. Unlike gradient-based optimization methods that iteratively minimize a cost function such as root mean square (RMS) error, this approach offers a deterministic, non-iterative solution, leading to improved performance for a faster execution.

Estimating all three parameters (α, Δ, and τ) simultaneously can present inconsistencies when only a limited number of μs-spaced samples are available. To improve robustness, we apply a tail-weighting strategy. In this approach, the decay waveform is augmented with K synthetic points at the baseline (≈0), effectively reinforcing the physical constraint that the signal should asymptotically approach the offset. In the least-squares fit, these additional points act as a regularizer, stabilizing the estimate of the DC offset, Δ, and reducing its correlation with τ. This allows the closed-form regression to first determine τ reliably, after which α and Δ are obtained from a simple 2×2 linear system. Using this method, the extracted lifetime, τ, matched theoretical values within a 0.5% error, while maintaining an execution time of 4 ms on the 32 MHz MCU.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An epidermally mounted wearable blood gas monitoring device, comprising:
an epidermally mounted substrate including:
a luminescent measurement sensor configured for computing a carbon dioxide blood gas level; and
a reflective sensor configured for computing an oxygen blood gas level;
an analog front end (AFE) coupled to the luminescent sensor and the reflective sensor;
a processor in communication with the AFE for simultaneous computation of transcutaneous carbon dioxide (PtcCO₂) and peripheral blood oxygen saturation (SpO₂); and
an epidermal engagement medium in gaseous communication with a sensed epidermal surface for detection of oxygen and carbon dioxide.

2. The device of claim 1 wherein the luminescent measurement sensor further comprises
a luminescent film responsive to a predetermined wavelength of light;
an illumination source configured to emit light in the predetermined wavelength;
the luminescent film responsive to the illumination source for emitting light of a responsive wavelength based on a concentration of carbon dioxide; and
a photodiode for receiving the emitted light and sending a signal to the AFE indicative of the emitted light.

3. The device of claim 2 wherein the luminescent film emits a CO₂ sensitive emission signal and a reference emission signal in response to transcutaneous carbon dioxide, further comprising comparing the CO₂ sensitive emission signal to the reference emission signal for determining the transcutaneous carbon dioxide.

4. The device of claim 2 wherein the luminescent film has dual luminophores, the dual luminophores selectively responsive to emit a CO₂ sensitive emission signal and a reference emission signal, wherein a difference in the CO₂ sensitive emission signal and the reference emission signal varies between an on and off state of the illumination source.

5. The device of claim 1 wherein the reflective sensor further comprises:
an illumination source having a wavelength based on a first hemoglobin sensitivity;
an illumination source having a wavelength based on a second hemoglobin sensitivity, the illumination sources for the first and second hemoglobin sensitivities directed towards a common epidermal location;
a photodetector disposed opposed from the common epidermal location and configured for receiving reflected light indicative of an oxygen saturation ratio.

6. The device of claim 5 where the oxygen saturation ratio is based on a ratio of reflected light corresponding to the first and second hemoglobin sensitivities, the first and second hemoglobin sensitivities differentiated based on an oxygenation state of hemoglobin molecules.

7. The device of claim 5 wherein the reflected light has an intensity in wavelengths corresponding to oxygenation states of hemoglobin in blood vessels beneath the common epidermal location, the oxygen saturation ratio based on absorption of hemoglobin corresponding to the respective oxygenation states.

8. The device of claim 1 wherein the epidermal engagement medium receives CO₂ from a gaseous emission of an epidermal surface and receives an indication of O₂ based on a blood vessel flow beneath the epidermal surface.

9. The device of claim 1 further comprising computing an oxygen level based on determining a carbon dioxide level erroneously contributing to the sensed SpO₂.

10. The device of claim 1 wherein the processor is further configured to:
compute the blood oxygen saturation based on both oxygen and carbon dioxide bound to hemoglobin;
determine, based on the carbon dioxide blood gas level, a portion of the computed oxygen blood gas level inaccurately attributed to carbon dioxide bound to hemoglobin; and
compute the peripheral blood oxygen saturation based on an adjustment for the inaccurately attributed carbon dioxide bound to hemoglobin.

11. The device of claim 1 wherein the luminescent measurement sensor and the reflective sensor are disposed in an adjacency on the epidermal engagement medium and coupled to the processor.

12. The device of claim 1 wherein the luminescent measurement sensor is configured for performing a dual-lifetime referencing (DLR) technique based on the predetermined wavelength in the range of 465-475 nm, and the responsive wavelength includes a 500-510 nm wavelength and a 585-595 nm wavelength.

13. The device of claim 1 wherein the reflective sensor is configured for emitting the illumination source in a wavelength of 655-665 nm and a wavelength of 845-855 nm, and the photodetector is responsive to the reflected light in a wavelength between 400 nm and 1000 nm.

14. A method for epidermally deployed blood gas monitoring device in a wearable appliance, comprising:
deploying an epidermally mounted substrate configured for affixation to a patient, the substrate in gaseous communication with a sensed epidermal surface for detection of oxygen and carbon dioxide;
computing, in a luminescent measurement sensor, a transcutaneous carbon dioxide (PtcCO₂) blood gas level;
determining, based on the carbon dioxide blood gas level, a portion of the computed oxygen blood gas level inaccurately attributed to carbon dioxide bound to hemoglobin.

15. The method of claim 14, further comprising rendering a true oxygen blood gas level in a patient having a diagnosis of a condition associated with elevated carbon dioxide levels.

16. The method of claim 14 wherein computing the carbon dioxide blood gas level further comprises deploying a luminescent film having dual luminophores, the dual luminophores selectively responsive to emit a CO₂ sensitive emission signal and a reference emission signal, and computing a difference between the CO₂ sensitive emission signal and the reference emission signal based on an ON and OFF state of the illumination source.

17. The method of claim 14 wherein computing the oxygen the reflective sensor further comprises:
activating an illumination source having a wavelength based on a first hemoglobin sensitivity;
activating an illumination source having a wavelength based on a second hemoglobin sensitivity, the illumination sources for the first and second hemoglobin sensitivities directed towards a common epidermal location;
receiving, from a photodetector disposed opposed from the common epidermal location, reflected light indicative of an oxygen saturation ratio.

18. A system for monitoring blood gases, comprising:
an epidermally mounted substrate including:
a luminescent measurement sensor configured for computing a carbon dioxide blood gas level; and
a reflective sensor configured for computing an oxygen blood gas level;
a processor for simultaneous computation of transcutaneous carbon dioxide (PtcCO₂) and peripheral blood oxygen saturation (SpO₂); and
an epidermal engagement medium in gaseous communication with a sensed epidermal surface for detection of oxygen and carbon dioxide.
